(19) United States
(12) Patent Application Publication
(10) Pub. No.: US 2024/0260727 A1
(43) Pub. Date: Aug. 8, 2024

(54) OPHTHALMOSCOPIC CONTACT LENSES

(71) Applicant: Alcon Inc., Fribourg (CH)

(72) Inventors: James M. Scott, Weatherford, TX (US); Patrick John Sadd, Colleyville, TX (US); David Cook, Dublin (IE); Sebastian Merx, Jena (DE); Joshua Coyne, Roundwood (IE); Kevin Coughlan, Dublin (IE)

(21) Appl. No.: 18/432,767

(22) Filed: Feb. 5, 2024

Related U.S. Application Data
(60) Provisional application No. 63/483,567, filed on Feb. 7, 2023.

(51) Int. Cl. A45C 11/00

(52) U.S. Cl. CPC A45C 11/005

(57) ABSTRACT

Certain embodiments disclose a contact lens holder including a rim forming a lens receptacle for receiving a lens and a flange integrally formed with and extending from the rim, flange comprising at least one tab, wherein at least one of a posterior surface of the flange and a posterior surface of the at least one tab comprises micro-structuring.

### BACKGROUND

The present disclosure relates to self-retaining ophthalmoscopic contact lens holders and contact lens assemblies for observation and/or surgical treatment of the vitreous or the retina.

Anatomically, the eye is divided into two distinct parts: the anterior segment and the posterior segment. The anterior segment includes the lens and extends from the outermost layer of the cornea (the corneal epithelium) to the posterior of the lens capsule. The posterior segment, which is much larger than the anterior segment, includes the portion of the eye behind the lens capsule. The posterior segment extends from the anterior hyaloids face to the retina, with which the posterior hyaloid face of the vitreous body is in direct contact, and further to the choroid and the posterior sclera.

The posterior segment includes the vitreous body, which is a clear, colorless, gel-like substance. The vitreous body gives the eye its globular shape and form, and comprises approximately two-thirds of the total volume of the eye. It is composed of 99% water and 1% collagen and sodium hyaluronate. The anterior boundary of the vitreous body is the anterior hyaloid face, which touches the posterior capsule of the lens. The posterior boundary of the vitreous body is the posterior hyaloid face, which is in contact with the retina. The vitreous body, unlike the aqueous humor in the anterior chamber, is not free-flowing and has normal anatomic attachment sites. These sites include the lens nerve head, the macula lutea, the vascular arcade, and the vitreous base, which is a 3-4 mm (millimeters) wide band that overlies the ora serrata. The vitreous body's major functions are to hold the retina in place, maintain the integrity and shape of the globe, absorb shock due to movement, and to give support for the posterior aspect of the lens.

In contrast to aqueous humor, the vitreous body is not continuously replaced, and it becomes more fluid with age through a process known as syneresis. Syneresis results in shrinkage of the vitreous body, which can exert pressure or traction on its normal attachment sites. If enough traction is applied, the vitreous body may pull itself from its retinal attachment and create a retinal tear or hole, which may necessitate surgical repair.

Vitreoretinal surgical procedures are used to treat many serious conditions of the posterior segment, including age-related macular degeneration (AMD), diabetic retinopathy, and diabetic vitreous hemorrhage, macular holes, retinal detachment, epiretinal membrane, cytomegalovirus (CMV) retinitis, and many other ophthalmic conditions. When performing surgery of the posterior segment of the eye, as in vitreoretinal surgery, it is typically necessary to view the anatomy of the eye with an operating microscope and an ophthalmoscopy lens designed to provide a clear image of the posterior segment. Generally, a standard operating microscope is able to view the structures of the anterior segment of the eye and the anterior portion of the posterior segment of the eye, but cannot adequately view the entire posterior segment of the eye because the natural optics of the eye (i.e., the cornea and the lens) prevent the operating microscope from focusing on some structures in the posterior segment of the eye (e.g., the retina). Therefore, in order to focus the operating microscope on structures such as the retina, an ophthalmoscopy lens with appropriate optical properties may be positioned between the eye and the microscope to compensate for the natural optics of the eye.

Direct ophthalmoscopy lenses, which create a virtual image within the eye, and indirect ophthalmoscopy lenses, which create a real image outside of the eye, are two lens types that have been used for observation of the posterior segment and as aids in the surgical treatment of the eye. Known lenses that are used in vitreoretinal surgery may suffer from less than desirable image quality due to loss of contrast and sharpness secondary to various optical phenomena, such as, by way of non-limiting example, defocusing, spherical aberration, coma, distortion, and chromatic aberration.

A challenge related to using surgical contact lenses placed on a patient's eye during an ophthalmic procedure is maintaining the position of the surgical contact lens during the procedure. One solution has been to have an assistant manually maintain the position of the surgical contact lens during the procedure. This solution requires the assistant to be highly trained. Further, any repositioning of the surgical contact lens by the assistant slows down the procedure being performed. Another solution has been to suture a portion of the surgical contact lens to the patient's eye. While this latter solution can successfully maintain the position of the surgical contact lens over the patient's eye, even a small suture is an injury to the patient's eye, and a less invasive process for maintaining the position of the surgical contact lens over the patient's eye would be beneficial.

### SUMMARY

The disclosure relates generally to, and encompasses, contact lens holders, apparatuses, and systems for use during an ophthalmoscopic surgery or procedure involving visualization of the posterior segment of the eye. The holders, apparatuses and systems disclosed herein provide increased stability and access space for insertion of surgical instruments as compared to traditional contact lenses. It will further be apparent that features of the present invention pertain to lens holders with or without lenses installed therein.

Certain embodiments disclose a contract lens holder including a rim forming a lens receptacle for receiving a lens and a flange integrally formed with and extending from the rim, flange comprising at least one tab, wherein at least one of a posterior surface of the flange and a posterior surface of the at least one tab comprises micro-structuring.

Certain embodiments disclose a contract lens assembly including a lens and a contact lens holder. The contact lens holder includes a rim forming a lens receptacle for receiving a lens and a flange integrally formed with and extending from the rim, flange comprising at least one tab, wherein at least one of a posterior surface of the flange and a posterior surface of the at least one tab comprises micro-structuring.

It is to be understood that both the foregoing general description and the following detailed description are exemplary and explanatory in nature and are intended to provide an understanding of the present disclosure without limiting the scope. In that regard, additional aspects, features, and advantages will be apparent to one skilled in the art from the following detailed description.

### BRIEF DESCRIPTION OF THE DRAWINGS

The accompanying drawings illustrate embodiments of the devices, systems, and methods disclosed herein and, together with the description, serve to explain the principles of the present disclosure.

FIG. 1 illustrates a perspective view of a contact lens holder, according to certain embodiments of the present disclosure.

FIG. 2 illustrates a perspective view of a contact lens assembly including a contact lens holder and contact lens, according to certain embodiments of the present disclosure.

FIG. 3 illustrates a partial cross-sectional side view of the contact lens assembly of FIG. 2, according to certain embodiments of the present disclosure.

FIG. 4 illustrates a top plan view of a contact lens holder, according to certain embodiments of the present disclosure.

FIGS. 5A-5B illustrate various views of a contact lens holder having an example surface micro-structuring, according to certain embodiments of the present disclosure.

FIGS. 6A-6C illustrate various blown up views of another example surface micro-structuring, according to certain embodiments of the present disclosure.

FIG. 7A illustrates a partial cross-sectional side view of a contact lens holder having an example surface macro-structuring, according to certain embodiments of the present disclosure.

FIG. 7B illustrates a blown up view of the surface macro-structuring of FIG. 7A, according to certain embodiments of the present disclosure.

FIG. 8 illustrates a partial cross-sectional side view of a contact lens holder having another example surface micro-structuring, according to certain embodiments of the present disclosure.

FIG. 9 illustrates a top plan view of a contact lens holder having an alternative tab configuration, according to certain embodiments of the present disclosure.

FIG. 10A illustrates a partial cross-sectional side view of a contact lens holder having another example surface micro-structuring, according to certain embodiments of the present disclosure.

FIG. 10B illustrates a blown up view of the surface micro-structuring of FIG. 10A, according to certain embodiments of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to certain embodiments may be combined with the features, components, and/or steps described with respect to other embodiments of the present disclosure. For simplicity, in some instances the same reference numbers are used throughout the drawings to refer to the same or like parts.

The present disclosure relates generally to ophthalmoscopic contact lens holders used in ophthalmic surgeries, such as vitreoretinal surgeries or other posterior segment surgeries, as well as ophthalmic clinical procedures. Certain embodiments herein provide a contact lens holder that may be used in conjunction with an aspheric lens to better visualize the interior of the eye, including the posterior segment. The combination of a contact lens holder (herein referred to as “lens holder”) and a contact lens positioned therein may be referred to as a contact lens assembly. In certain embodiments, a lens holder and a contact lens positioned within the lens holder are separate components, in which case, the contact lens can be removed and replaced with a different contact lens, if desired. In certain other embodiments, a lens holder and a contact lens are attached and not separable.

In certain embodiments, a lens holder includes at least one flange having at least one tab that extends from the flange. The at least one tab includes surface micro-structuring that improve self-retention of the lens holder on the eye, without the use of sutures or without the lens holder being manually held. A self-retaining lens holder refers to a lens holder that is able to self-stabilize and remain on the eye during a procedure without intervention (e.g., without being held manually or being sutured to the eye).

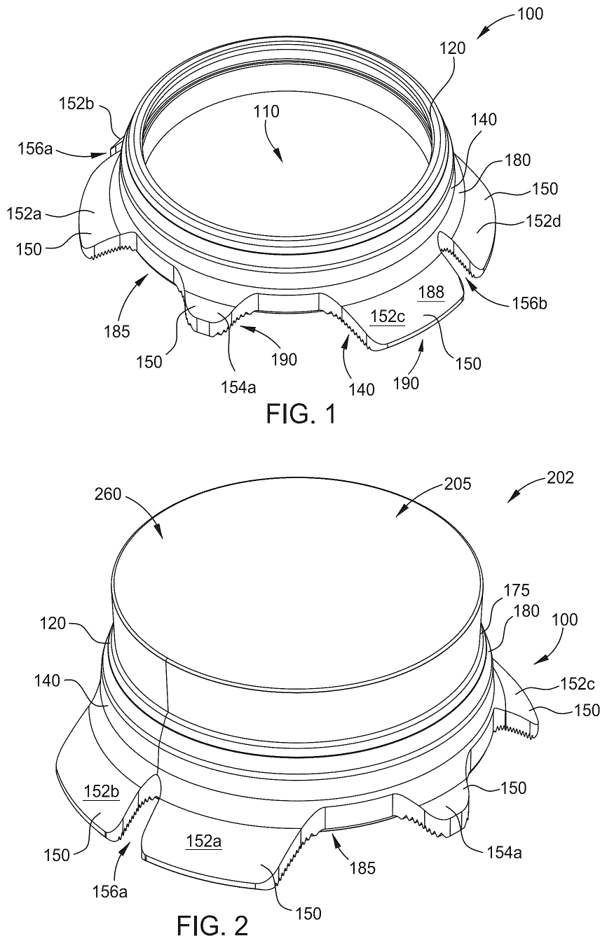

FIG. 1 illustrates a perspective view of a lens holder 100, according to certain embodiments of the present disclosure. Though the lens holder 100 shown in FIG. 1 is configured for use in ophthalmologic surgeries, such as vitreoretinal surgery, the lens holder 100 may be used in any ophthalmological context, including diagnosis, treatment, etc. The lens holder 100 is configured to receive a lens (as shown in FIG. 2) that may be used in combination with a surgical microscope, a slit lamp, or any other ophthalmic viewing device to view the interior of an eye. For example, a surgical microscope may be spaced from and cooperate with the lens inside the lens holder 100 for capturing light rays exiting the eye through the cornea and passing through the lens. The surgical microscope can focus such light rays to create an image of, for example, the retina and the vitreous body.

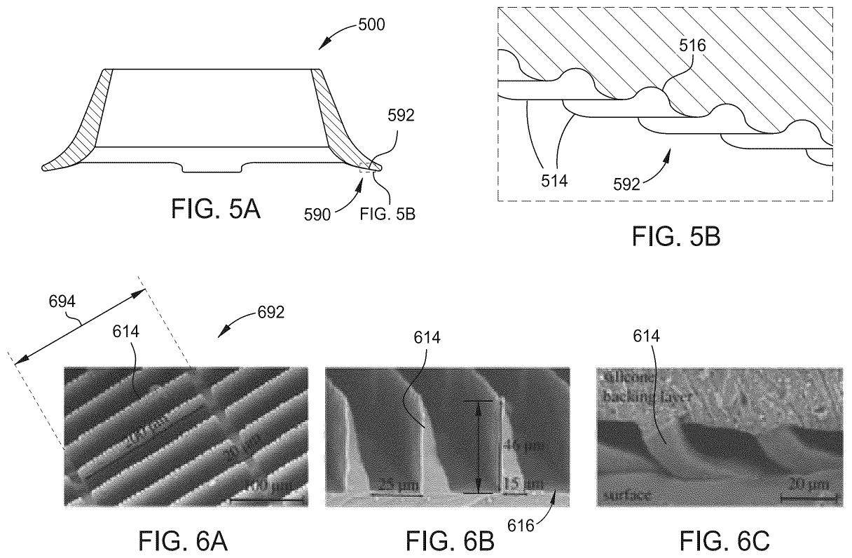

In the pictured embodiment, the lens holder 100 comprises a one-piece device including integrally formed components. The lens holder 100 includes a central lens receptacle 110 for holding a lens, such as of the plano-concave, convex-concave (meniscus), or bi-concave type, as shown in FIG. 2. The lens receptacle 110 is formed and circumferentially surrounded by a cylindrical rim 120. A circular flange 140, which is integrally formed with the rim 120, extends from and angles away from the rim 120. In the embodiments of FIG. 1, a plurality of tabs 150 project outward from the flange 140. At least one of the tabs 150 and/or the flange 140 have surface micro- or macro-structuring to provide enhanced stability, as shown in FIGS. 5A-8B.

In the embodiment shown in FIG. 1, the tabs 150 comprises four major tabs 152 and two minor tabs 154. As shown, a first major tab 152a and a second major tab 152b are separated by a first relief space 156a. For reference, the location of the first relief space 156a corresponds to the 12:00 o'clock position. The first major tab 152a and the second major tab 152b are located in a superior quadrant of the lens holder 100 (between a 10:30 o'clock position and a 1:30 o'clock position). A third major tab 152c and a fourth major tab 152d are separated by a second relief space 156b, and are located in an inferior quadrant (between a 4:30 o'clock position and a 7:30 o'clock position) of the lens holder 100.

As shown, a first minor tab 154a is located between the first major tab 152a and the third major tab 152c at approximately a 9:00 o'clock position. A second minor tab 154b (not shown in the view provided by FIG. 1) is located between the second major tab 152b and the fourth major tab 152d at approximately the 3:00 o'clock position. In the shown embodiments, the major tabs 152a, 152b, 152c, 154d are the same first size, and the minor tabs 154a, 154b are the same second size. However, it will be appreciated that the tabs 150 may be shaped, sized, and/or numbered differently.

As shown, the minor tabs 154a, 154b, are smaller than the major tabs 152 to leave some operating space for the surgeon but still help provide additional stability to the lens holder 100. Further, between each minor tab 154 and a major tab 152, a relief space is provided. An example of such a relief space is provided as relief space 185 between minor tab 154s and major tab 152a. The relief spaces between each minor tab 154 and a neighboring major tab 152 is typically where a surgeon would insert various surgical instruments, such as trocar cannulas into which probes (e.g., irrigation probes, laser probes, aspiration probes, illumination probes, etc.) are inserted during surgery. For example, a typical vitreoretinal surgery requires placement of three trocar cannulas to provide ports for entry of surgical instruments and fluid into the eye. Typically, one port is used for fluid infusion and two ports are used for instrument insertion (e.g., one active port and one illumination port). Trocar cannulas are typically positioned such that they are spaced 3.5-4.5 mm away from the limbus of the eye (where the pars plana is located), which has an average diameter of approximately 11.7 mm, to avoid damage to the ciliary processes and the ora serrata.

As such, the number of tabs, the dimension of the various tabs, including minor tabs 154, major tabs 152, and/or the various relief spaces provided herein are configured and sized such as to provide a surgeon with the necessary operating space for surgical instrument insertion etc., while maximizing the lens holder's 100 contact surface with the eye, thereby enhancing the lens holder's self-retention capabilities.

Each of the tabs 150 includes an anterior tab surface and a posterior tab surface. For example, major tab 152c comprises an anterior tab surface 188 and a posterior tab surface 190. Each posterior tab surface has a curved shape substantially corresponding to the scleral curvature of the eye, thereby allowing the tabs 150 to sit approximately flush against the eye.

In some embodiments, the lens receptacle 110 may be sized to have an active diameter of approximately 14 mm (millimeters), which can accommodate a lens of up to 14 mm in diameter. The 14 mm diameter is larger than a typical dilated pupil. The 14 mm diameter of the lens receptacle is large enough to facilitate adequate light passing through a lens positioned in the lens receptacle 110 but also small enough to limit interference with a surgeon's hand during an ophthalmological procedure. It will be appreciated, however, that the diameter may be larger or smaller for various applications.

FIG. 2 illustrates a perspective view of a contact lens assembly 202 including the lens holder 100 of FIG. 1 and a contact lens 205, according to certain embodiments. The lens 205 includes an aspheric anterior optic surface or base profile 260 and a posterior optic surface (not shown in the view provided in FIG. 2). The posterior optic surface, in some embodiments, has a curved spherical shape substantially corresponding to the corneal curvature of an average human cornea.

In some embodiments, the anterior optic surface 260 includes an anti-reflective or non-reflective coating to reduce reflective glare for improved visualization. A non-reflective or anti-reflective coating may improve the ability to capture digital video or image frames reducing or eliminating artifacts in a 2-dimensional microscope view.

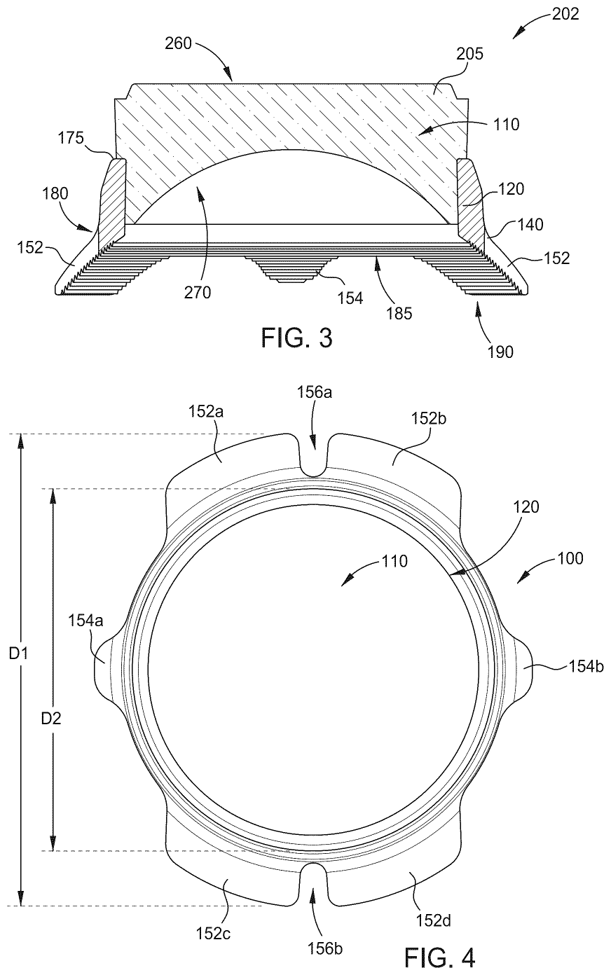

Note that, the lens holder 100 is configured to accommodate lenses having different heights or diopter powers. For example, although in the pictured embodiment of FIGS. 2-3, the lens 205 proximally extends above an edge 175 of the rim 120, in embodiments where a shorter lens is used, the edge 175 of the rim 120 may extend above the anterior optic surface 260.

FIG. 3 illustrates a cross-sectional view of the lens holder 100 and lens 205. With reference to FIG. 3, the circular flange 140 encircles and extends away from the rim 120 at an angle, forming a peripheral flared region surrounding a base circumference of the lens receptacle 110. As shown, the flange 140 forms an integral extension of the rim 120, and extends radially from the lens receptacle 110 such that, at least in certain embodiments, if the surgical contact lens holder 100 is centrally positioned over a cornea of an eye, the flange 140 would extend onto the sclera of the eye.

In some embodiments, the flange 140 is shaped, configured to be thin enough, and made from certain material, such as to provide some pliancy and flexibility. In some embodiments, the flange 140 may be thinner or wider than rim 120. In some embodiments, the flange 140 has a thickness between about 0.5 mm and about 1.5 mm, such as between about 0.55 mm and about 1.45 mm such as between about 0.6 mm and about 1.4 mm, such as between about 0.65 mm and about 1.35 mm, such as between about 0.7 mm and about 1.3 mm, such as between about 0.75 and about 1.25 mm, such as between about 0.8 mm and about 1.2 mm, such as between about 0.85 mm and about 1.15 mm, such as between about 0.9 mm and about 1.1 mm, such as between about 0.95 mm and about 1.05 mm. In some embodiments, the thickness of the flange 140 may depend on the hardness of a material of the flange 140. For example, with a less rigid material, the flange 140 may have a thicker thickness. Alternatively, with a more rigid material, the flange may have a reduced thickness to increase pliancy and flexibility thereof.

In some embodiments, the flange 140 and/or the tabs 150 are made from a hydrophobic silicone rubber material or other suitable elastomeric material. This material may be soft enough to allow a surgeon to easily clip or cut unwanted portions of the flange 140 and/or tabs 150, if desired. In some embodiments, this material may have a shore A hardness between about 30 Shore A and about 95 Shore A, such as between about 30 Shore A and about 80 Shore A, such as about 70 Shore A. In certain embodiments, the flange may be semi-rigid or rigid. In certain embodiments, the flange 140 may be shaped and configured to be transparent enough to provide for visualization through the flange to observe, by way of non-limiting example, underlying tissue, vessels, air bubbles, and/or bleeding. In alternate embodiments, the flange may be semi-transparent or opaque.

The flange 140 includes an anterior flange surface 180 and a posterior flange surface 185. The posterior flange surface 185 is shaped and configured to have a different curvature from that of the posterior optic surface 270. For example, in the pictured embodiment shown in FIG. 3, the posterior flange surface 185 has a curved shape substantially corresponding to the curvature (e.g., corneal and/or scleral curvature) of an average human eye, thereby allowing the flange 140 to sit approximately flush against the eye while the lens 205 sits approximately flush against the cornea of the eye. This combination of varying curvatures conforming to different portions of an average human eye tends to center and stabilize the lens holder over the cornea of the eye. In the embodiments of FIG. 3, posterior flange surface 185 includes micro-structuring, such as the micro-structuring shown in FIG. 6A. However, any other types of micro-structuring, or macro-structuring, provided herein may be used on the posterior flange surface.

As discussed above, the tabs 150 extend from the flange 140 at an angle and are shaped to conform to the curvature of an average curvature of the human eye. The tabs 150 may be shaped in any of a variety of shapes, including, by way of non-limiting example, tabs, triangles, oblongs (e.g., curved oblongs), and finger-like extensions. Furthermore, the tabs 150 may be sized according to ocular anatomy and/or a modulus of a material of the tabs 150.

As discussed, the lens holder 100 is configured with a number of self-retaining features, thereby making the lens holder 100 a self-retaining lens holder, allowing it to be used in ophthalmic procedures in a hands-free manner. For example, one of such self-retaining features is the large surface area of the posterior flange surface 185 and the posterior tab surfaces 190 that contact the surface of the eye (i.e., the cornea and/or the sclera), thereby increasing the stability of the lens holder 100. In some embodiments, the posterior tab surfaces 190 each have a surface area (e.g., a contact area) of between about 10 square millimeters (mm²) and about 30 mm², such as about 20 mm². In such embodiments, the surface area of both posterior tab surfaces 190 is between about 20 mm² and about 60 mm², such as about 40 mm². In some embodiments, the lens holder 100 includes more than 200 mm² of surface contact area. Another self-retaining/balancing feature is the surface micro- or macro-structuring on the posterior flange surface 185 or on one or more posterior tab surfaces 190, which also increase stability. With surface micro-structuring in particular, the surface area of both posterior tab surfaces 190 may be between about 60 mm²and about 80 mm² or more in certain embodiments. Generally, micro-structuring can increase the surface area between about 50% and about 200%, depending on micro-structuring design, which itself may depend on the hardness of the material of flange 140.

Yet another self-retaining/balancing feature is the curvature of the posterior flange surface 185 and the posterior tab surfaces 190. For example, as shown in FIG. 3, the posterior flange surface 185 and the posterior tab surfaces 190 comprise interior concave surfaces that are configured to have a radius of curvature that is the same or substantially the same as the curvature of an average eye. In particular, the curvature of the posterior flange surface 185 and the posterior tab surfaces 190 may be the same or substantially the same as the scleral curvature of an average eye (e.g., average eye in a certain age group or for a certain gender, ethnicity, conditions, etc.). In some embodiments, the posterior flange surface 185 and the posterior tab surfaces 190 may have apical radii R of approximately 11.0 to 12.0 mm. In some embodiments, the curvature of the posterior optic surface 270 may be the same or substantially the same as the corneal curvature of an average eye (e.g., average eye in a certain age group or for a certain gender, ethnicity, conditions, etc.).

FIG. 4 illustrates a top plan view of lens holder 100, according to certain embodiments of the present disclosure. The tabs are sized and positioned to maximize stability while also maximizing the available working space for insertion of surgical instruments such as, by way of non-limiting example, trocar cannulas positioned near the lens holder 100. In the pictured embodiment, the lens holder 100 includes six tabs (four major tabs 152a, 152b, 152c, 152d and two minor tabs 154a, 154b). A maximum diameter D1 of the lens holder 100 with the major tabs 154 may be 18.50 mm. In the embodiment shown, an inner diameter D2 of the rim 120 may be 14.00 mm. However, in certain other embodiments, the maximum diameter D1 and inner diameter D2 may be larger or smaller for various applications. For example, in some embodiments, the maximum diameter D1 may be 17.00 mm or less. In various embodiments, it may be desirable that the inner diameter D2 match the diameter of a lens being received in the lens receptacle 110.

The surgical contact lens holder 100 may be positioned on the cornea with the use of an interface solution, such as, by way of non-limiting example, a viscoelastic or another similar agent. Example viscoelastic fluids that may be suitable for this purpose include, without limitation: Viscoat® viscoelastic—40,000 centipoise (“cps”), DicCoVisc® viscoelastic—75,000 cps, Healon® viscoelastic—50,000-4,000,000 cps, EYEFILL® SC viscoelastic—400,000 cps, and AMVISC® viscoelastic—55,700 cps. The interface solution functions to keep the cornea hydrated and to create or increase the shear forces between the ocular tissue and the lens holder 100. For example, posterior optic surface of the contact lens, the posterior flange surface 185, and/or the posterior tab surfaces 190 may generate shear forces with the interface solution between the surface of the eye and the lens holder 100/lens 205, thereby providing additional stability to the lens holder 100 during use.

To increase the shear forces between the ocular tissue and the lens holder 100 even further and provide greater stability, the embodiments described in relation to FIGS. 5A through 9 provide one or more examples of surface micro-structuring (e.g., variegated or textured surfaces) and macro-structuring (e.g., removal of material to form perforations therein) for the tabs 150, the posterior flange surface 185, and/or the posterior tab surfaces 190.

FIG. 5A illustrates a partial cross-sectional side view of a contact lens holder 500 having a surface micro-structuring 592 on a posterior tab surface 590 of one or more tabs, according to certain embodiments. FIG. 5B illustrates a blown-up view of the surface micro-structuring 592 shown in FIG. 5A. As shown, the micro-structuring 592 includes a plurality of ridges 514 separated by recesses 516. In the example of FIG. 5B, ridges 514 and recesses 516 are in the form of peaks and troughs that have half-circle or half-round shapes. The depth, width, and shape of the ridges 514 and/or the recesses 516 may be selected for maximized surface contact adhesion to optimize stability of the lens holder 500, e.g., by optimizing the shear forces between the ocular tissue and the posterior tab surfaces 590. Further, the depth, width, and shape of the ridges 514 and/or the recesses 516 may depend on a modulus of a material of the ridges 514 and/or the recesses 516. In some embodiments, the ridges 514 may have a width between about 0.05 mm and about 0.3 mm, such as between about 0.1 mm and about 0.25 mm, such as between about 0.15 mm and about 0.2 mm, such as about 0.16 mm. In some embodiments, the ridges 514 may have a height (and/or the recesses 516 may have a depth) between about 0.1 mm and about 0.4 mm, such as between about 0.15 mm and about 0.35 mm, such as between about 0.2 mm and about 0.3 mm, such as about 0.22 mm or about 0.25 mm. In some embodiments, the ridges 514 may have a cross-sectional area between about 0.01 mm² and about 0.03 mm², such as between about 0.15 mm² and about 0.25 mm², such as about 0.02 mm².

FIGS. 6A-6C illustrate various blown-up views of another example surface micro-structuring 692 on a posterior tab surface of one or more tabs of a lens holder, according to embodiments of the present disclosure. As shown, the micro-structuring 692 includes at least substantially triangular ridges 614 that are separated by flat recesses, each recess providing a gap between two neighboring ridges 614. In other embodiments, triangular ridges 614 may not be separated by flat recesses, and may instead resemble a saw tooth contour. In the example of FIGS. 6A-6C, the ridges 614 are shaped like right triangles; however other types of triangular shapes are also within the scope of the disclosure. In the example of FIG. 6A, micro-structuring 692 includes a number of rows and columns of ridges 614, where each ridge 614 has a length 694 equal to about 200 μm. As shown in FIG. 6A, the neighboring columns of ridges 614 are separated by a gap having a length of about 20 μm. In the example of FIG. 6B, ridges 614 have a height of about 46 μm, a width of about 15 μm, and are separated by a separation width of about 25 μm. The dimensions provided herein in relation to FIGS. 6A-6C are merely examples and other dimensions are within the scope of the disclosure.

Ridges 614 may be made of flexible material, such as silicone, and flex when in contact with the surface of the eye. FIG. 6C shows an example of ridges 614 flexing when a lens holder having ridges 614 is placed on the surface of the eye and pressure is applied thereto.

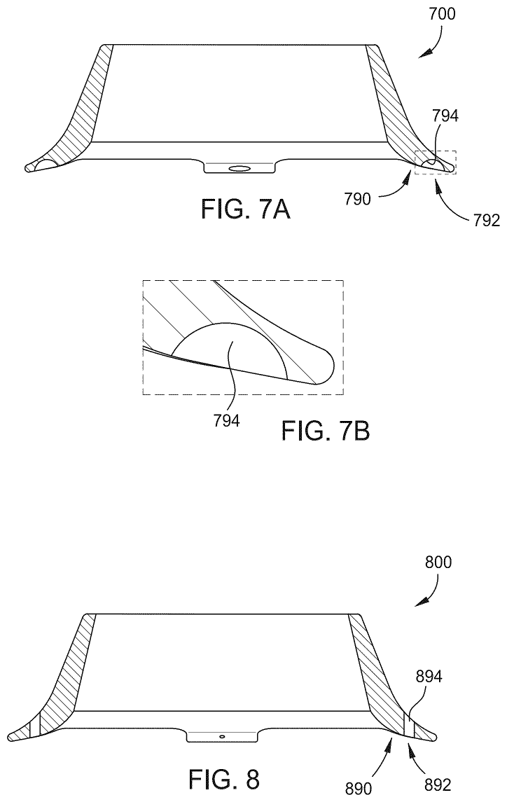

FIG. 7A illustrates a partial cross-sectional side view of a contact lens holder 700 having a surface macro-structuring 792, according to certain embodiments of the present disclosure. FIG. 7B illustrates a blown-up view of the surface macro-structuring 792 shown in FIG. 7A. As shown, the surface macro-structuring 792 comprises at least one suction cup or recess 794 on the posterior surface 790 of one or more of the tabs to increase flexibility of the lens holder 700 for suction thereof onto ocular tissue. The size and geometry of the at least one suction recess 794 may be selected for optimizing surface contact adhesion to improve stability of the lens holder 700 such as by optimizing negative pressure traction, suction forces, or shear forces between the ocular tissue and the posterior tab surfaces 790. Further, more than one suction recess may be used to create additional suction force between lens holder 700 and the surface of the eye.

FIG. 8 illustrates a partial cross-sectional side view of a contact lens holder 800 having a surface micro-structuring 892, according to certain embodiments of the present disclosure. As shown, the surface micro-structuring 892 comprises at least one channel 894 extending through one or more of the tabs from the posterior surface of the tabs to the anterior surface of the tabs. The size and geometry of the at least one channel 894 may be selected for optimizing surface contact adhesion to improve stability of the lens holder 800 such as by optimizing capillary action attachment, pressure, or shear forces between the ocular tissue and the tabs 850, particularly in the presence of a viscoelastic fluid. Further, more than one channel may be used to create additional suction force between lens holder 800 and the surface of the eye.

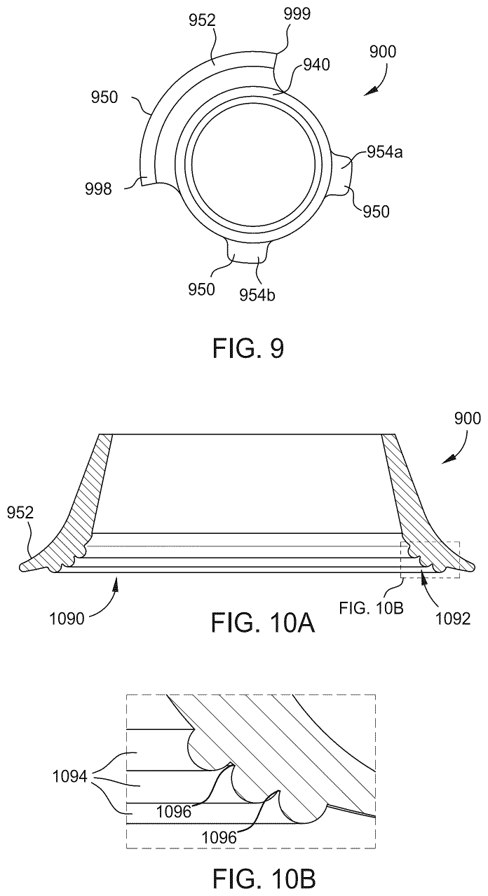

FIG. 9 illustrates a top plan view of a contact lens holder 900 having an alternative tab configuration, according to certain embodiments of the present disclosure. The contact lens holder 900 may include any of the aforementioned features, except that the tabs 950 are configured as shown.

As shown, the contact lens holder 900 comprises a major tab 952 extending peripherally from the flange 940 along an entire quadrant of the flange 940, or more (from approximately a 9:00 to a 12:00 position). A first minor tab 954a extends peripherally from the flange 940 at a location approximately opposite a first end 998 of the major tab 950 (at approximately a 3:00 position), and a second minor tab 954b extends peripherally from the flange 940 at a location approximately opposite a second end 999 of the major tab 950 (at approximately a 6:00 position).

FIG. 10A illustrates a partial cross-sectional side view of a lens holder, such as the lens holder 900 of FIG. 9, having a surface micro-structuring 1092 on the posterior surface 1090 of a major tab, such as the major tab 950, according to certain embodiments of the present disclosure. FIG. 10B illustrates a blown-up view of the surface micro-structuring 1092 shown in FIG. 10A. As shown, the surface micro-structuring 1092 comprises a ridge pattern having a plurality of (e.g., three) ridges 1094. As shown, in the embodiments of FIG. 10A-10B, each ridge has the shape of a smooth, half-circle or a half-round contour. In the example of FIG. 10B, there is a slight gap 1096 between each two neighboring ridges 1094. The size and geometry of the ridges 1094 may be selected for optimizing surface contact adhesion to improve stability of the lens holder 700, e.g., by optimizing the shear forces between the ocular tissue and the posterior tab surfaces 790. Generally, the dimensions of ridges 1094 are based on a modulus of a material thereof. In the example of FIGS. 10A and 10B, ridges 1095 have a radius between about 0.25 mm and about 1.25 mm, such as between about 0.5 mm and about 1.0 mm. In some examples, the size and/or shape of the gaps 1096 between neighboring ridges 1094 is dependent on a radius of a tool or machinery utilized for fabricating the lends holder, e.g., a diamond tool having a minimum tip radius of about 10 μm (other tip radii for other size/shape of the gaps are also contemplated).

The various lens holders described herein are self-retaining such that the lens holders can be used during ophthalmic procedures without the aid of an assistant's handle. However, in certain embodiments, the lens holder embodiments may be used in conjunction with a handle to provide increased control and/or maneuverability of the lens holder on the eye.

As previously mentioned, some or all of the flange 140 and tabs 150 may be composed of a hydrophobic silicone rubber material. However, portions of the lens holders described herein may also be suitably formed from any of a variety of biocompatible materials, including, by way of non-limiting example, cyclo olefin copolymers, PMMA (Poly(methyl methacrylate)), Zeonex, Topas, silicon rubber, Acrysof, Polycarbonate (PC), acrylic, epoxy, polysulfone (PS), polyphenylsulfone (PPSU), Polyetherimide (PEI), and/or Polyethylene terephthalate (or poly(ethylene terephthalate) (PET). In some embodiments, the various components of the lens holders, including the flange, the rim, and the tabs, are formed from the same biocompatible material. In other embodiments, the various components of the lens holders are formed from different biocompatible materials.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

### EXAMPLE EMBODIMENTS

### Embodiment 1

A contact lens holder, comprising: a rim forming a lens receptacle for receiving a lens; and a flange integrally formed with and extending from the rim, flange comprising at least one tab, wherein at least one of a posterior surface of the flange and a posterior surface of the at least one tab comprises micro-structuring.

### Embodiment 2

The contact lens holder of Embodiment 1, wherein the flange has a curvature corresponding to a scleral and/or corneal curvature of an average eye.

### Embodiment 3

The contact lens holder of Embodiment 1, wherein at least one of the at least one tab and the flange is made of hydrophobic silicon rubber.

### Embodiment 4

The contact lens holder of Embodiment 1, wherein the lens receptacle has an inner diameter of 14 mm and the flange has a diameter of 18.5 mm.

### Embodiment 5

The contact lens holder of Embodiment 1, wherein the at least one tab includes an adhesive.

### Embodiment 6

A contact lens assembly comprising: a lens; and a contact lens holder, comprising: a rim forming a lens receptacle holding the lens; and a flange integrally formed with and extending from the rim, flange comprising at least one tab, wherein at least one of a posterior surface of the flange and a posterior surface of the at least one tab comprises micro-structuring.

What is claimed is:

1. A contact lens holder, comprising:
a rim forming a lens receptacle for receiving a lens; and
a flange integrally formed with and extending from the rim, flange comprising at least one tab, wherein at least one of a posterior surface of the flange and a posterior surface of the at least one tab comprises micro-structuring.

2. The contact lens holder of claim 1, wherein the micro-structuring comprises a plurality of ridges separated by a plurality of recesses.

3. The contact lens holder of claim 2, wherein the plurality of the ridges and the plurality of recesses have half-circles or half-round shapes.

4. The contact lens holder of claim 1, wherein the micro-structuring comprises triangular ridges separated by flat recesses.

5. The contact lens holder of claim 4, wherein the triangular ridges comprises rows and columns of ridges, wherein neighboring columns of ridges are separated by a gap.

6. The contact lens holder of claim 1, wherein the micro-structuring on the at least one tab comprises one or more suction recesses.

7. The contact lens holder of claim 1, wherein the micro-structuring on the at least one tab comprises one or more channels that extend from the posterior surface of the at least one tab to an anterior surface of the at least one tab.

8. The contact lens holder of claim 1, wherein the at least one tab comprises a plurality of tabs with various sizes.

9. The contact lens holder of claim 8, wherein the plurality of tabs comprise a first set of tabs including two major tabs in a superior quadrant of the flange and a second set of tabs including another two major tabs in an inferior quadrant of the flange.

10. The contact lens holder of claim 9, wherein the first set of tabs and the second set of tabs are of a same size.

11. The contact lens holder of claim 9, wherein there is a relief space between each of the two tabs in the first set of tabs and each of the other two tabs in the second set of tabs.

12. The contact lens holder of claim 9, wherein the plurality of tabs comprise a third set tabs including two minor tabs, wherein each of the minor tabs is situation between a major tab from the first set of tabs and a major tab from the second set of tabs.

13. The contact lens holder of claim 9, wherein the third set of tabs are smaller in size than the first and the second sets of tabs.

14. The contact lens holder of claim 8, wherein the plurality of tabs comprise three tabs, including a major tab and two minor tabs, wherein the major tab extends peripherally along an entire quadrant of the flange, and wherein the two minor tabs include a first minor tab extending peripherally at a first location approximately opposite a first end of the major tab and a second minor tab extends peripherally at a second location approximately opposite a second end of the major tab.

15. The contact lens holder of claim 1, wherein at least one of a posterior surface of the flange and a posterior surface of the at least one tab comprises viscoelastic material.
